(19) United States
(12) Patent Application Publication
(10) Pub. No.: US 2022/0354347 A1
(43) Pub. Date: Nov. 10, 2022

(54) MEDICAL SUPPORT ARM AND MEDICAL SYSTEM

(71) Applicant: SONY GROUP CORPORATION, TOKYO (JP)

(72) Inventors: NAOKI NISHIMURA, TOKYO (JP); MASARU USUI, TOKYO (JP)

(21) Appl. No.: 17/640,642

(22) PCT Filed: Sep. 4, 2020
(86) PCT No.: PCT/JP2020/033676

§ 371 Date: Mar. 4, 2022

(30) Foreign Application Priority Data
Sep. 12, 2019 (JP) 2019-165850

(51) Int. Cl. A61B 1/00; A61B 1/313; A61B 1/04

(52) U.S. Cl. CPC A61B 1/00149; A61B 1/000095; A61B 1/00006; A61B 1/3132; A61B 1/04; A61B 2034/301

(57) ABSTRACT

A medical support arm includes: a support arm that supports an endoscope; an actuator that drives the support arm; a measurement unit that measures a load applied to the actuator; a generation unit that generates three-dimensional information in a body into which the endoscope is inserted; and a correction unit that corrects the three-dimensional information on a basis of the measured load.

### FIELD

The present disclosure relates to a medical support arm and a medical system.

### BACKGROUND

In endoscopic surgery, an image of the abdominal cavity of a patient is captured using an endoscope, and surgery is performed while a captured image captured by the endoscope is displayed on a display.

For example, Patent Literature 1 discloses a technology of controlling an operation of a support arm for an endoscope so that an observation target is always positioned within a region of a captured image when the endoscope is inserted into the human body and operated.

### CITATION LIST

### Patent Literature

Patent Literature 1: JP 2018-75218 A

### SUMMARY

### Technical Problem

In laparoscopic surgery, since the surgery is performed while establishing pneumoperitoneum, a position (trocar position) of an insertion port of an endoscope may be changed during the surgery. Therefore, it is not easy to control the support arm so that the endoscope maintains a state suitable for surgery.

Therefore, the present disclosure proposes a medical support arm and a medical system capable of appropriately controlling movement of a support arm.

### Solution to Problem

To solve the above problem, a medical support arm according to the present disclosure includes: a support arm that supports an endoscope; an actuator that drives the support arm; a measurement unit that measures a load applied to the actuator; a generation unit that generates three-dimensional information in a body into which the endoscope is inserted; and a correction unit that corrects the three-dimensional information on a basis of the measured load.

### BRIEF DESCRIPTION OF DRAWINGS

FIG. 1 is a diagram illustrating a configuration of a robot arm that supports an endoscope.

FIG. 2 is a view illustrating an example of an in-vivo image captured by the endoscope.

FIG. 3 is a diagram illustrating a state in which an operator inserts an instrument in a state in which pneumoperitoneum is sufficient.

FIG. 4 is a diagram illustrating a state in which the operator inserts the instrument in a state in which pneumoperitoneum is insufficient.

FIG. 5 is a diagram illustrating a state in which carbon dioxide gas escapes during surgery and the abdominal wall descends.

FIG. 6 is a diagram illustrating an example of a schematic configuration of an endoscopic surgery system to which the technology according to the present disclosure can be applied.

FIG. 7 is a block diagram illustrating an example of functional configurations of a camera head and a camera control unit (CCU) illustrated in FIG. 6.

FIG. 8 is a schematic diagram illustrating an appearance of a support arm device according to the present embodiment.

FIG. 9 is a schematic diagram illustrating a configuration of an oblique-viewing endoscope according to an embodiment of the present disclosure.

FIG. 10 is a schematic diagram illustrating the oblique-viewing endoscope and a forward-viewing endoscope in comparison.

FIG. 11 is a block diagram illustrating an example of a configuration of a medical observation system according to an embodiment of the present disclosure.

FIG. 12 is a diagram illustrating a specific configuration example of a robot arm device according to an embodiment of the present disclosure.

FIG. 13 is a diagram illustrating a state in which the endoscope captures an image of an area of interest.

FIG. 14 is a diagram illustrating a state in which the endoscope captures an image of the area of interest.

FIG. 15 is a flowchart illustrating an example of control processing for holding the endoscope in the area of interest of the operator.

FIG. 16 is a flowchart illustrating an example of processing for controlling a position of the endoscope so as to track a surgical instrument during surgery.

### DESCRIPTION OF EMBODIMENTS

Hereinafter, embodiments of the present disclosure will be described in detail with reference to the drawings. Note that, in each of the following embodiments, the same reference signs denote the same portions, and an overlapping description will be omitted.

Further, the present disclosure will be described in the following order.

1. Introduction
1-1. Purpose and the like of Present Embodiment
1-2. Outline of Present Embodiment

2. Configuration of Medical System
2-1. First Configuration Example (Endoscope System)
2-2. Specific Configuration Example of Support Arm Device
2-3. Specific Configuration Example of Endoscope
2-4. Second Configuration Example (Medical Observation System)

3. Operation of Medical System
3-1. Example of Control of Arm (Control Based on SLAM)
3-2. Example of Control of Arm (Control in Consideration of Tracking of Surgical Instrument)

4. Modification

5. Conclusion

<<1. Introduction>>

<1-1. Purpose and the Like of Present Embodiment>

In endoscopic surgery, an image of the abdominal cavity of a patient is captured using an endoscope (rigid endoscope), and surgery is performed while a captured image captured by the endoscope is displayed on a display. In laparoscopic surgery, since the surgery is performed while establishing pneumoperitoneum (for example, while inflating the belly with CO₂), an insertion port (that is, the origin position of the endoscope) is changed during the surgery. Furthermore, in the laparoscopic surgery, in some cases, a scope (lens barrel) attached to a camera head may be changed during the surgery or dirt attached to a distal end of the scope may be removed, and thus the scope is removed from a trocar and then inserted again. Therefore, in a case where a control device is caused to control an operation of a support arm (hereinafter, also referred to as an endoscope arm) of the endoscope so that a position (for example, a position of the distal end of the scope) of the endoscope is suitable for the surgery, the control device may erroneously recognize a current position of the endoscope. For example, at the time of inserting a surgical instrument, an operation of pulling the endoscope arm to a trocar portion is performed to avoid collision with the instrument. However, at this time, there is a possibility that the endoscope arm cannot sufficiently pull the endoscope, and the endoscope collides with the instrument.

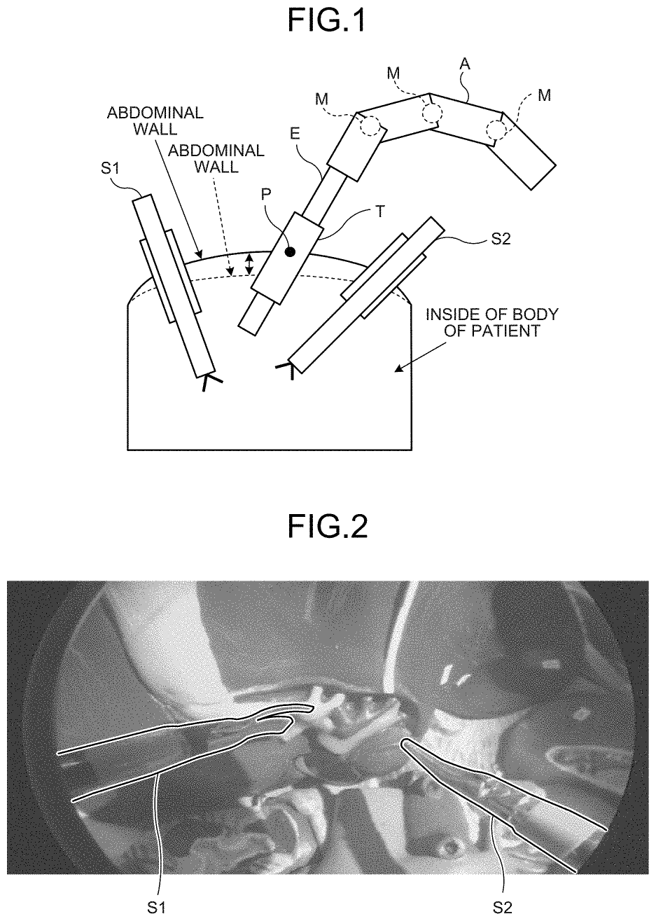

This problem will be specifically described with reference to the drawings. FIG. 1 is a diagram illustrating a configuration of a robot arm A that supports an endoscope E. The endoscope E is connected to the robot arm A (one aspect of a computer-aided surgery system). The endoscope E is, for example, a rigid endoscope. Note that, in the present embodiment, the endoscope includes a scope (lens barrel) and a camera head, but the endoscope does not have to necessarily include the camera head. For example, only a portion corresponding to the scope (lens barrel) may be regarded as the endoscope. The robot arm of the present embodiment supports, for example, the camera head to which the scope (lens barrel) is attached. A motor M for controlling an arm of each axis is arranged inside the robot arm A. The endoscope E is inserted into the body of the patient through a trocar T, and captures an image of an area in which the operator is interested. Here, the trocar T is an instrument called a medical puncture instrument. Note that the surgical instrument (for example, instruments S1 and S2 illustrated in FIG. 1) is also inserted into the body of the patient through the trocar. The operator performs laparoscopic surgery while viewing the image captured by the endoscope E.

In the laparoscopic surgery, a space is required to secure an imaging view of the rigid endoscope, and the surgery is performed while injecting carbon dioxide gas into the body (establishing pneumoperitoneum) to secure the space. A broken line portion in FIG. 1 indicates a change of the abdominal wall during surgery, and a position of the abdominal wall is changed during surgery. In a case where pneumoperitoneum is insufficient, carbon dioxide gas is inserted into the body again to secure a clear view.

FIG. 2 is a view illustrating an example of an in-vivo image captured by the endoscope E. The robot arm A of the present embodiment may be an autonomous/semi-autonomous robot arm. Therefore, for example, a control device (for example, a processor inside or outside the robot arm A) that controls the robot arm A recognizes an image of the instrument S1 and/or the instrument S2, and autonomously holds the endoscope E in an area that the operator desires to view, such that the image of the instrument S1 and/or the instrument S2 can be captured.

For example, the control device of the robot arm A performs simultaneous localization and mapping (SLAM) in the body while calculating a distance to a point of interest in the image obtained by the endoscope with a trocar point P illustrated in FIG. 1 as the origin. In the present embodiment, the trocar point means a boundary point between the inside and the outside of the body. In order to calculate the distance to the point of interest in the image, the control device may use information from a monocular camera or information from a stereo camera. Furthermore, the control device may use information from a distance measuring sensor such as a time-of-flight (ToF) sensor. Note that the robot arm A may include sensors such as a monocular camera and a distance measuring sensor in advance, or may be configured so that these sensors can be attached and detached.

As described above, in the laparoscopic surgery, the surgery is performed while establishing pneumoperitoneum in order to secure the imaging view of the endoscope. Since the carbon dioxide gas injected into the body escapes from the body during surgery, the abdominal wall is always changed during surgery, and thus it is difficult to perform the entire sensing again in the body during surgery. Therefore, it is assumed that it is difficult to regenerate an in-vivo environment map (that is, three-dimensional information in the body) created by SLAM from scratch in real time according to the change of the state of the abdominal wall. As a result, the in-vivo environment map created by SLAM differs from the actual state depending on the pneumoperitoneum state in the body.

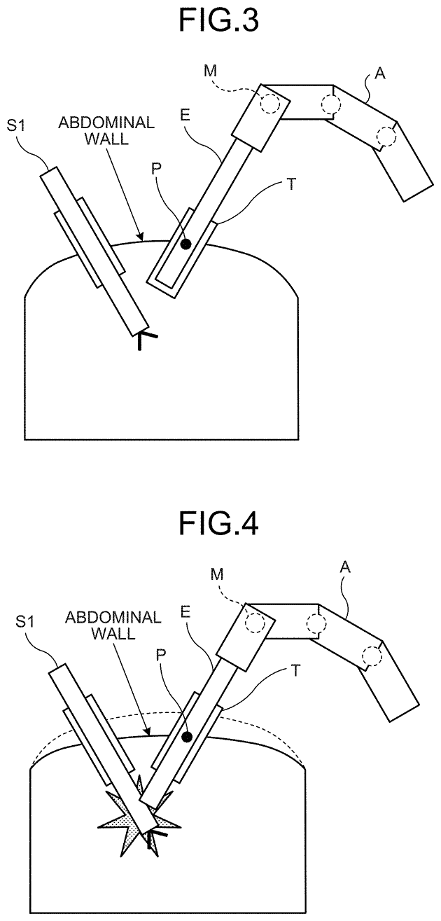

In order to facilitate understanding of the problem of the present embodiment, a case where the surgical instrument is inserted into the body will be described as an example. In order to avoid contact between the surgical instrument and the endoscope when the instrument is inserted into the body, in many cases, the endoscope E is pulled to the position of the trocar T on the basis of a trigger (or mode selection) from the operator. The operator then inserts the instrument into the body. FIG. 3 is a diagram illustrating a state in which the operator inserts the instrument S1 in a state in which pneumoperitoneum is sufficient.

For example, it is assumed that SLAM (or initial sensing for SLAM) is performed in a state in which the pneumoperitoneum is sufficient as illustrated in FIG. 3. Further, it is assumed that the operator tries to insert the instrument S1 into the body in a state in which the pneumoperitoneum is sufficient. In this case, since the control device controls the robot arm A on the basis of information of the in-vivo environment map generated by SLAM, the endoscope E can be sufficiently pulled to the position of the trocar T as illustrated in FIG. 3. As a result, a sufficient space for inserting the instrument S1 is secured.

However, as described above, the state of the abdominal wall is constantly changed. FIG. 4 is a diagram illustrating a state in which the operator inserts the instrument S1 in a state in which the pneumoperitoneum is insufficient. For example, it is assumed that the carbon dioxide gas in the body escapes as illustrated in FIG. 4, for example, after SLAM is performed in a state in which the pneumoperitoneum is sufficient as illustrated in FIG. 3. Further, it is assumed that the operator tries to insert the instrument S1 into the body in a state in which the pneumoperitoneum is insufficient. In this case, the trocar point P set as the origin is lower than that assumed in the in-vivo environment map. Even in a case where the control device controls the robot arm A so as to pull the endoscope E to the position of the trocar T in this state, the robot arm A cannot sufficiently pull the endoscope E. In this case, the endoscope E is held at a position where the instrument S1 and the endoscope E may come into contact with each other.

<1-2. Overview of Present Embodiment>

Therefore, in the present embodiment, the in-vivo environment map (three-dimensional information in the body) is corrected according to the state of the abdominal wall. More specifically, the control device of the robot arm A measures a load applied to the motor M that drives the arm supporting the endoscope, and corrects the in-vivo map on the basis of the measured load.

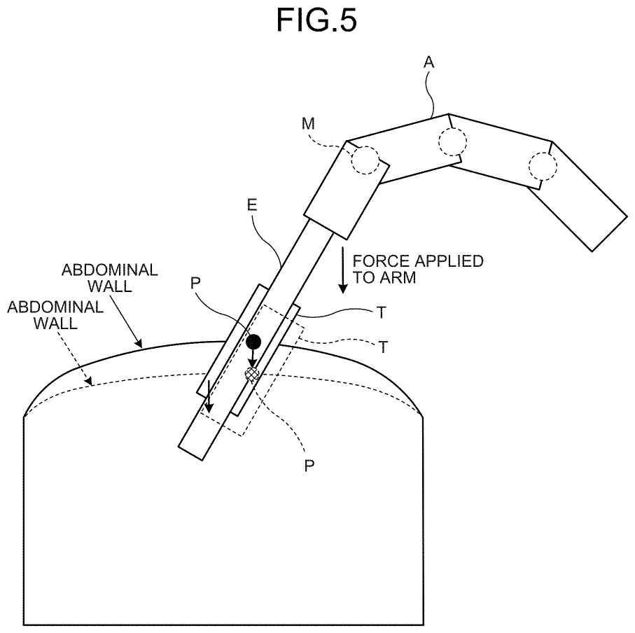

FIG. 5 is a diagram illustrating a state in which the carbon dioxide gas escapes during surgery and the abdominal wall descends. Broken line portions in FIG. 5 indicate positions of the abdominal wall and the trocar T after the carbon dioxide gas has escaped from the body. Once the abdominal wall descends, the position of the trocar T also descends accordingly. When the position of the trocar T descends, a downward force is applied to the endoscope E from the trocar T. The load of the motor M is increased because the downward force is further applied in addition to a force in a gravity direction. The control device can estimate an actual current position of the trocar point P on the basis of the change in load. Therefore, the control device corrects the in-vivo map on the basis of the change in load.

As a result, the robot arm A can be controlled on the basis of the highly accurate in-vivo map (three-dimensional information in the body), such that the robot arm A can hold the endoscope E at a position suitable for surgery.

Although the outline of the present embodiment has been described above, a medical system including the medical support arm (for example, the robot arm A) of the present embodiment will be described in detail below.

<<2. Configuration of Medical System>>

Before describing an operation of the medical system of the present embodiment, a configuration (device configuration and functional configuration) of the medical system will be described. For the medical system of the present embodiment, several configuration examples can be considered.

<2-1. First Configuration Example (Endoscope System)>

First, a configuration of an endoscope system will be described as an example of the medical system of the present embodiment.

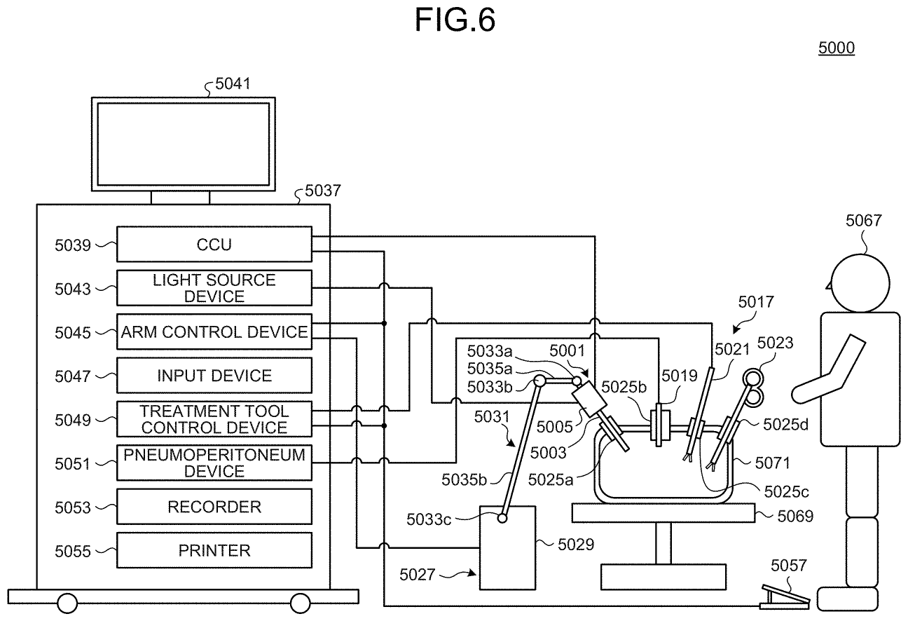

FIG. 6 is a diagram illustrating an example of a schematic configuration of an endoscopic surgery system 5000 to which the technology according to the present disclosure can be applied. In the example of FIG. 6, a state in which an operator (for example, a doctor) 5067 is performing surgery on a patient 5071 on a patient bed 5069 by using the endoscopic surgery system 5000 is illustrated. As illustrated, the endoscopic surgery system 5000 includes an endoscope 5001, other surgical tools 5017, a support arm device 5027 that supports the endoscope 5001, and a cart 5037 on which various devices for endoscopic surgery are mounted.

The endoscope 5001 corresponds to, for example, the endoscope E illustrated in FIGS. 1 to 5, and the support arm device 5027 corresponds to, for example, the robot arm A illustrated in FIGS. 1 to 5.

In endoscopic surgery, instead of cutting and opening the abdominal wall, a plurality of cylindrical puncture instruments called trocars 5025a to 5025d puncture the abdominal wall. Then, a lens barrel 5003 of the endoscope 5001 and the other surgical tools 5017 are inserted into the body cavity of the patient 5071 through the trocars 5025a to 5025d. In the illustrated example, as the other surgical tools 5017, a pneumoperitoneum tube 5019, an energy treatment tool 5021, and forceps 5023 are inserted into the body cavity of the patient 5071. Furthermore, the energy treatment tool 5021 is a treatment tool for incision and peeling of tissue, vascular closure, or the like by using a high-frequency current or ultrasonic vibration. However, the illustrated surgical tools 5017 are merely an example, and various surgical tools generally used in endoscopic surgery, such as tweezers and a retractor may be used as the surgical tools 5017.

An image of a surgical site in the body cavity of the patient 5071 captured by the endoscope 5001 is displayed on a display device 5041. The operator 5067 performs treatment such as resection of an affected part by using the energy treatment tool 5021 or the forceps 5023 while viewing the image of the surgical site displayed on the display device 5041 in real time. Note that, although not illustrated, the pneumoperitoneum tube 5019, the energy treatment tool 5021, and the forceps 5023 are supported by the operator 5067, an assistant, or the like during surgery.

(Support Arm Device)

The support arm device 5027 includes an arm portion 5031 extending from a base portion 5029. In the illustrated example, the arm portion 5031 includes joint portions 5033a, 5033b, and 5033c and links 5035a and 5035b, and is driven under the control of an arm control device 5045. The arm portion 5031 supports the endoscope 5001 and controls a position and a posture of the endoscope 5001. As a result, it is possible to stably fix the position of the endoscope 5001.

(Endoscope)

The endoscope 5001 includes the lens barrel 5003 in which a region corresponding to a predetermined length from a distal end is inserted into the body cavity of the patient 5071, and a camera head 5005 connected to a proximal end of the lens barrel 5003. In the illustrated example, the endoscope 5001 configured as a so-called rigid endoscope including the rigid lens barrel 5003 is illustrated, but the endoscope 5001 may be configured as a so-called flexible endoscope including the flexible lens barrel 5003.

An opening portion into which an objective lens is fitted is provided at the distal end of the lens barrel 5003. A light source device 5043 is connected to the endoscope 5001, and light generated by the light source device 5043 is guided to the distal end of the lens barrel by a light guide extending inside the lens barrel 5003, and is emitted toward an observation target in the body cavity of the patient 5071 via the objective lens. Note that the endoscope 5001 may be a forward-viewing endoscope, an oblique-viewing endoscope, or a side-viewing endoscope.

An optical system and an imaging element are provided inside the camera head 5005, and reflected light (observation light) from the observation target is collected on the imaging element by the optical system. The observation light is photoelectrically converted by the imaging element, and an electric signal corresponding to the observation light, that is, an image signal corresponding to an observation image is generated. The image signal is transmitted to a camera control unit (CCU) 5039 as raw data. Note that the camera head 5005 has a function of adjusting a magnification and a focal length by appropriately driving the optical system.

Note that, for example, a plurality of imaging elements may be provided in the camera head 5005 in order to support stereoscopic viewing (3D display) or the like. In this case, a plurality of relay optical systems are provided inside the lens barrel 5003 in order to guide the observation light to each of the plurality of imaging elements.

(Various Devices Mounted on Cart)

The CCU 5039 is implemented by a central processing unit (CPU), a graphics processing unit (GPU), and the like, and integrally controls operations of the endoscope 5001 and the display device 5041. Specifically, the CCU 5039 performs, on the image signal received from the camera head 5005, various types of image processing for displaying an image based on the image signal, such as development processing (demosaic processing), for example. The CCU 5039 provides the image signal subjected to the image processing to the display device 5041. Furthermore, the CCU 5039 transmits a control signal to the camera head 5005 to control driving thereof. The control signal can include information regarding imaging conditions such as a magnification and a focal length.

The display device 5041 displays an image based on the image signal subjected to the image processing by the CCU 5039 under the control of the CCU 5039. In a case where the endoscope 5001 supports high-resolution imaging such as 4K (the number of horizontal pixels 3840×the number of vertical pixels 2160) or 8K (the number of horizontal pixels 7680×the number of vertical pixels 4320), and/or in a case where the endoscope supports 3D display, a display device capable of high-resolution display and/or a display device capable of 3D display can be used as the display device 5041 for each case. In a case where the display device supports the high-resolution imaging such as 4K or 8K, a further immersive feeling can be obtained by using, as the display device 5041, a display device with a size of 55 inches or more. Furthermore, a plurality of display devices 5041 having different resolutions and sizes may be provided depending on the application.

The light source device 5043 is implemented by a light source such as a light emitting diode (LED), for example, and supplies, to the endoscope 5001, irradiation light for capturing an image of the surgical site.

The arm control device 5045 is implemented by, for example, a processor such as a CPU, and is operated according to a predetermined program to control driving of the arm portion 5031 of the support arm device 5027 according to a predetermined control method. The arm control device 5045 corresponds to the control device (for example, the control device for the robot arm A) that controls the support arm of the present embodiment. Note that the CCU 5039 can also be regarded as the control device of the present embodiment.

An input device 5047 is an input interface for the endoscopic surgery system 5000. A user can input various types of information or instructions to the endoscopic surgery system 5000 via the input device 5047. For example, the user inputs various types of information regarding surgery, such as physical information of a patient and information regarding a surgical procedure of the surgery, via the input device 5047. Furthermore, for example, the user inputs an instruction to drive the arm portion 5031, an instruction to change the imaging conditions (a type of the irradiation light, a magnification, a focal length, and the like) of the endoscope 5001, an instruction to drive the energy treatment tool 5021, and the like via the input device 5047.

The type of the input device 5047 is not limited, and the input device 5047 may be various known input devices. As the input device 5047, for example, a mouse, a keyboard, a touch panel, a switch, a foot switch 5057, a lever, and/or the like can be applied. In a case where a touch panel is used as the input device 5047, the touch panel may be provided on a display surface of the display device 5041.

Alternatively, the input device 5047 is a device worn by the user, such as a glasses-type wearable device or a head-mounted display (HMD), and various inputs are performed according to a gesture or a gaze of the user detected by these devices. Furthermore, the input device 5047 includes a camera capable of detecting movement of the user, and various inputs are performed according to a gesture or a gaze of the user detected from a video captured by the camera. Furthermore, the input device 5047 includes a microphone capable of collecting user's voice, and various inputs are performed by voice via the microphone. As described above, the input device 5047 is configured to be able to input various types of information in a non-contact manner, and thus, in particular, a user (for example, the operator 5067) belonging to a clean area can operate a device belonging to an unclean area in a non-contact manner. In addition, since the user can operate the device without releasing his/her hand from the held surgical tool, the convenience of the user is improved.

A treatment tool control device 5049 controls driving of the energy treatment tool 5021 for cauterization and incision of tissue, vascular closure, or the like. A pneumoperitoneum device 5051 feeds gas into the body cavity of the patient 5071 via the pneumoperitoneum tube 5019 in order to inflate the body cavity for the purpose of securing a clear view for the endoscope 5001 and securing a working space for the operator. A recorder 5053 is a device capable of recording various types of information regarding surgery. A printer 5055 is a device capable of printing various types of information regarding surgery in various formats such as text, images, or graphs.

Hereinafter, a particularly characteristic configuration of the endoscopic surgery system 5000 will be described in more detail.

(Support Arm Device)

The support arm device 5027 includes the base portion 5029 which is a base, and the arm portion 5031 extending from the base portion 5029. The support arm device 5027 may include a control device that functions as the arm control device 5045 and/or the CCU 5039. The support arm device 5027 corresponds to the support arm (for example, the robot arm A) of the present embodiment. The arm portion 5031 may be regarded as the support arm of the present embodiment.

In the illustrated example, the arm portion 5031 includes the plurality of joint portions 5033a, 5033b, and 5033c and the plurality of links 5035a and 5035b connected by the joint portion 5033b, but in FIG. 6, the configuration of the arm portion 5031 is illustrated in a simplified manner for the sake of simplicity. In actual implementation, the shapes, the numbers, and the arrangements of the joint portions 5033a to 5033c and the links 5035a and 5035b, directions of rotation axes of the joint portions 5033a to 5033c, and the like can be appropriately set so that the arm portion 5031 has a desired degree of freedom. For example, the arm portion 5031 can be suitably configured to have six degrees of freedom or more. As a result, since the endoscope 5001 can be freely moved within a movable range of the arm portion 5031, the lens barrel 5003 of the endoscope 5001 can be inserted into the body cavity of the patient 5071 from a desired direction.

Actuators are provided in the joint portions 5033a to 5033c, and the joint portions 5033a to 5033c are configured to be rotatable around predetermined rotation axes by driving of the actuators. The driving of the actuator is controlled by the arm control device 5045, whereby a rotation angle of each of the joint portions 5033a to 5033c is controlled, and the driving of the arm portion 5031 is controlled. As a result, it is possible to control the position and the posture of the endoscope 5001. At this time, the arm control device 5045 can control the driving of the arm portion 5031 by various known control methods such as a power control or a position control.

For example, the operator 5067 may appropriately perform an operation input via the input device 5047 (including the foot switch 5057) to cause the arm control device 5045 to appropriately control the driving of the arm portion 5031 according to the operation input, thereby controlling the position and the posture of the endoscope 5001. With this control, the endoscope 5001 at the distal end of the arm portion 5031 can be moved from an arbitrary position to an arbitrary position and then fixedly supported at the position after the movement. Note that the arm portion 5031 may be operated by a so-called master-slave method. In this case, the arm portion 5031 (slave) can be remotely operated by the user via the input device 5047 (master console) installed at a place away from an operating room or in the operating room.

Furthermore, in a case where the power control is applied, the arm control device 5045 may perform a so-called power assist control of receiving an external force from the user and driving the actuator of each of the joint portions 5033a to 5033c so that the arm portion 5031 is smoothly moved according to the external force. As a result, when the user moves the arm portion 5031 while directly touching the arm portion 5031, the arm portion 5031 can be moved with a relatively small force. Therefore, it is possible to more intuitively move the endoscope 5001 with a simpler operation, and the convenience of the user can be improved.

Here, in general, in endoscopic surgery, the endoscope 5001 is supported by a doctor called scopist. However, the use of the support arm device 5027 enables more reliable fixation of the position of the endoscope 5001 without manual operation, and thus, it is possible to stably obtain the image of the surgical site and smoothly perform the surgery.

Note that the arm control device 5045 is not necessarily provided in the cart 5037. Furthermore, the arm control device 5045 is not necessarily one device. For example, the arm control device 5045 may be provided in each of the joint portions 5033a to 5033c of the arm portion 5031 of the support arm device 5027, and a driving control for the arm portion 5031 may be implemented by a plurality of arm control devices 5045 cooperating with each other.

(Light Source Device)

The light source device 5043 supplies the irradiation light for capturing an image of the surgical site to the endoscope 5001. The light source device 5043 includes, for example, a white light source implemented by an LED, a laser light source, or a combination thereof. At this time, in a case where the white light source is implemented by a combination of RGB laser light sources, an output intensity and an output timing of each color (each wavelength) can be controlled with high accuracy, and thus, white balance adjustment of the captured image can be performed in the light source device 5043. Furthermore, in this case, the observation target is irradiated with laser light from each of the RGB laser light sources in a time division manner and the driving of the imaging element of the camera head 5005 is controlled in synchronization with a timing of the irradiation, such that it is also possible to capture an image corresponding to each of RGB in a time division manner. With this method, a color image can be obtained without providing a color filter in the imaging element.

Furthermore, the driving of the light source device 5043 may be controlled so as to change the intensity of light to be output every predetermined time. The driving of the imaging element of the camera head 5005 is controlled in synchronization with a timing of the change of the intensity of the light to acquire images in a time division manner and images are combined, such that it is possible to generate a high-dynamic-range image without so-called underexposure and overexposure.

Furthermore, the light source device 5043 may be configured to be able to supply light in a predetermined wavelength band corresponding to special light observation. In the special light observation, for example, so-called narrow band imaging, in which an image of a predetermined tissue such as a blood vessel in a mucosal epithelial layer is captured with high contrast by radiating light in a narrower band than irradiation light (that is, white light) used at the time of normal observation, by using wavelength dependency of light absorption in a body tissue, is performed. Alternatively, in the special light observation, fluorescence observation for obtaining an image by fluorescence generated by irradiation with excitation light may be performed. In the fluorescence observation, for example, fluorescence from a body tissue can be observed by irradiating the body tissue with excitation light (autofluorescence observation), or a fluorescent image can be obtained by locally injecting a reagent such as indocyanine green (ICG) into a body tissue and irradiating the body tissue with excitation light corresponding to a fluorescence wavelength of the reagent. The light source device 5043 can be configured to be able to supply narrow band light and/or excitation light corresponding to such special light observation.

(Camera Head and CCU)

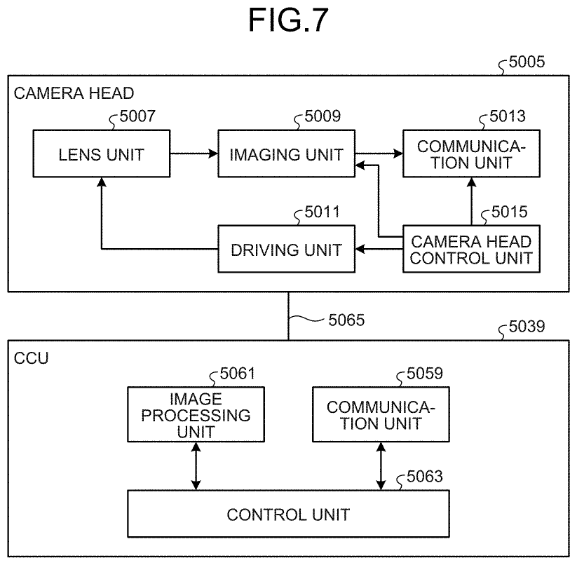

Functions of the camera head 5005 and the CCU 5039 of the endoscope 5001 will be described in more detail with reference to FIG. 7. FIG. 7 is a block diagram illustrating an example of functional configurations of the camera head 5005 and the CCU 5039 illustrated in FIG. 6.

Referring to FIG. 7, the camera head 5005 includes a lens unit 5007, an imaging unit 5009, a driving unit 5011, a communication unit 5013, and a camera head control unit 5015 as the functions thereof. Further, the CCU 5039 includes a communication unit 5059, an image processing unit 5061, and a control unit 5063 as the functions thereof. The camera head 5005 and the CCU 5039 are connected by a transmission cable 5065 so as to be bidirectionally communicable.

First, the functional configuration of the camera head 5005 will be described. The lens unit 5007 is an optical system provided at a portion at which the camera head 5005 is connected to the lens barrel 5003. The observation light taken in from the distal end of the lens barrel 5003 is guided to the camera head 5005 and is incident on the lens unit 5007. The lens unit 5007 is implemented by combining a plurality of lenses including a zoom lens and a focus lens. An optical characteristic of the lens unit 5007 is adjusted so as to concentrate the observation light on a light receiving surface of the imaging element of the imaging unit 5009. In addition, the zoom lens and the focus lens are configured to be movable on an optical axis thereof in order to adjust a magnification and a focal point of the captured image.

The imaging unit 5009 includes the imaging element and is arranged at a subsequent stage of the lens unit 5007. The observation light having passed through the lens unit 5007 is collected on the light receiving surface of the imaging element, and an image signal corresponding to the observation image is generated by photoelectric conversion. The image signal generated by the imaging unit 5009 is provided to the communication unit 5013.

For example, a complementary metal oxide semiconductor (CMOS) image sensor that has a Bayer array and is capable of color capturing is used as the imaging element included in the imaging unit 5009. Note that, as the imaging element, for example, an imaging element that can support the high-resolution imaging of 4K or more may be used. Since the high-resolution image of the surgical site is obtained, the operator 5067 can grasp a state of the surgical site in more detail, and can progress the surgery more smoothly.

Furthermore, the imaging element included in the imaging unit 5009 includes a pair of imaging elements for acquiring image signals for the right eye and the left eye corresponding to 3D display, respectively. As the 3D display is performed, the operator 5067 can more accurately grasp a depth of a living tissue in the surgical site. Note that, in a case where the imaging unit 5009 is configured as a multi-plate type, a plurality of lens units 5007 are provided corresponding to the respective imaging elements.

Furthermore, the imaging unit 5009 does not have to be necessarily provided in the camera head 5005. For example, the imaging unit 5009 may be provided immediately behind the objective lens inside the lens barrel 5003.

The driving unit 5011 is implemented by an actuator, and moves the zoom lens and the focus lens of the lens unit 5007 by a predetermined distance along the optical axis under the control of the camera head control unit 5015. As a result, the magnification and the focal point of the image captured by the imaging unit 5009 can be appropriately adjusted.

The communication unit 5013 is implemented by a communication device for transmitting and receiving various types of information to and from the CCU 5039. The communication unit 5013 transmits the image signal obtained from the imaging unit 5009 as raw data to the CCU 5039 via the transmission cable 5065. At this time, in order to display the captured image of the surgical site with low latency, the image signal is preferably transmitted by optical communication. This is because, at the time of surgery, the operator 5067 performs surgery while observing the state of the affected part in the captured image, and thus, for safer and more reliable surgery, it is required to display a moving image of the surgical site in real time as much as possible. In a case where optical communication is performed, a photoelectric conversion module that converts an electric signal into an optical signal is provided in the communication unit 5013. The image signal is converted into the optical signal by the photoelectric conversion module and then transmitted to the CCU 5039 via the transmission cable 5065.

Furthermore, the communication unit 5013 receives a control signal for controlling driving of the camera head 5005 from the CCU 5039. The control signal includes, for example, information regarding imaging conditions such as information for specifying a frame rate of the captured image, information for specifying an exposure value at the time of imaging, and/or information for specifying the magnification and the focal point of the captured image. The communication unit 5013 provides the received control signal to the camera head control unit 5015. Note that the control signal from the CCU 5039 may also be transmitted by optical communication. In this case, the photoelectric conversion module that converts an optical signal into an electric signal is provided in the communication unit 5013, and the control signal is converted into an electric signal by the photoelectric conversion module and then provided to the camera head control unit 5015.

Note that the imaging conditions such as the frame rate, the exposure value, the magnification, and the focal point are automatically set by the control unit 5063 of the CCU 5039 on the basis of the acquired image signal. That is, the endoscope 5001 has a so-called auto exposure (AE) function, an auto focus (AF) function, and an auto white balance (AWB) function.

The camera head control unit 5015 controls the driving of the camera head 5005 on the basis of the control signal received from the CCU 5039 via the communication unit 5013. For example, the camera head control unit 5015 controls driving of the imaging element of the imaging unit 5009 on the basis of the information for specifying the frame rate of the captured image and/or the information for specifying light exposure at the time of imaging. Furthermore, for example, the camera head control unit 5015 appropriately moves the zoom lens and the focus lens of the lens unit 5007 via the driving unit 5011 on the basis of the information for specifying the magnification and the focal point of the captured image. The camera head control unit 5015 may further have a function of storing information for identifying the lens barrel 5003 or the camera head 5005.

Note that, as the lens unit 5007, the imaging unit 5009, and the like are arranged in a sealed structure having high airtightness and waterproofness, the camera head 5005 can have resistance to autoclave sterilization processing.

Next, the functional configuration of the CCU 5039 will be described. The communication unit 5059 is implemented by a communication device for transmitting and receiving various types of information to and from the camera head 5005. The communication unit 5059 receives the image signal transmitted from the camera head 5005 via the transmission cable 5065. At this time, as described above, the image signal can be suitably transmitted by optical communication. In this case, for optical communication, a photoelectric conversion module that converts an optical signal into an electric signal is provided in the communication unit 5059. The communication unit 5059 provides the image signal converted into the electric signal to the image processing unit 5061.

Furthermore, the communication unit 5059 transmits a control signal for controlling the driving of the camera head 5005 to the camera head 5005. The control signal may also be transmitted by optical communication.

The image processing unit 5061 performs various types of image processing on the image signal that is raw data transmitted from the camera head 5005. Examples of the image processing include various types of known signal processing such as development processing, image quality enhancement processing (band emphasis processing, super-resolution processing, noise reduction (NR) processing, image stabilization processing, and/or the like), and/or enlargement processing (electronic zoom processing). Furthermore, the image processing unit 5061 performs wave detection processing on the image signal for performing the AE, the AF, and the AWB.

The image processing unit 5061 is implemented by a processor such as a CPU or a GPU, and the processor is operated according to a predetermined program, whereby the above-described image processing and wave detection processing can be performed. Note that, in a case where the image processing unit 5061 is implemented by a plurality of GPUs, the image processing unit 5061 appropriately divides information related to the image signal, and the plurality of GPUs perform the image processing in parallel.

The control unit 5063 performs various types of controls related to capturing of the image of the surgical site performed by the endoscope 5001 and display of the captured image. For example, the control unit 5063 generates a control signal for controlling the driving of the camera head 5005. At this time, in a case where the imaging condition is input by the user, the control unit 5063 generates the control signal on the basis of the input from the user. Alternatively, in a case where the endoscope 5001 has the AE function, the AF function, and the AWB function, the control unit 5063 appropriately calculates an optimum exposure value, focal length, and white balance according to a result of the wave detection processing performed by the image processing unit 5061, and generates the control signal.

Furthermore, the control unit 5063 causes the display device 5041 to display the image of the surgical site on the basis of the image signal subjected to the image processing by the image processing unit 5061. At this time, the control unit 5063 recognizes various objects in the image of the surgical site by using various image recognition technologies. For example, the control unit 5063 can recognize the surgical tool such as forceps, a specific site in the living body, bleeding, mist at the time of using the energy treatment tool 5021, and the like by detecting an edge shape, color, and the like of the object included in the image of the surgical site. When displaying the image of the surgical site on the display device 5041, the control unit 5063 superimposes various types of surgery support information on the image of the surgical site by using the recognition result. The surgery support information is superimposed and presented to the operator 5067, such that the surgery can be more safely and reliably performed.

The transmission cable 5065 connecting the camera head 5005 and the CCU 5039 is an electric signal cable supporting electric signal communication, an optical fiber supporting optical communication, or a composite cable thereof.

Here, in the illustrated example, wired communication is performed using the transmission cable 5065, but wireless communication may be performed between the camera head 5005 and the CCU 5039. In a case where wireless communication is performed between the camera head 5005 and the CCU 5039, it is not necessary to install the transmission cable 5065 in the operating room, and thus, a situation in which movement of a medical staff in the operating room is hindered by the transmission cable 5065 can be eliminated.

Hereinabove, an example of the endoscopic surgery system 5000 to which the technology according to the present disclosure can be applied has been described. Note that, here, the endoscopic surgery system 5000 has been described as an example, but a system to which the technology according to the present disclosure can be applied is not limited to such an example. For example, the technology according to the present disclosure may be applied to a flexible endoscope system for examination or a microscopic surgery system.

<2-2. Specific Configuration Example of Support Arm Device>

The medical system (computer-aided surgery system) of the present embodiment includes a support arm device. Hereinafter, a specific configuration example of the support arm device according to an embodiment of the present disclosure will be described in detail. Note that the use of the support arm device as described below is not limited to medical use.

The support arm device described below is an example configured as a support arm device that supports an endoscope at a distal end of an arm portion, but the present embodiment is not limited to such an example. Furthermore, in a case where the support arm device according to the embodiment of the present disclosure is applied to the medical field, the support arm device according to the embodiment of the present disclosure can function as a medical support arm device.

Note that the support arm device described below can not only be applied to the endoscopic surgery system 5000, but also be applied to other medical systems. It is a matter of course that the support arm device described below can also be applied to a system other than medical systems. Furthermore, as a control unit (control device) that performs processing of the present embodiment is installed in the support arm device, the support arm device itself may be regarded as the medical system of the present embodiment.

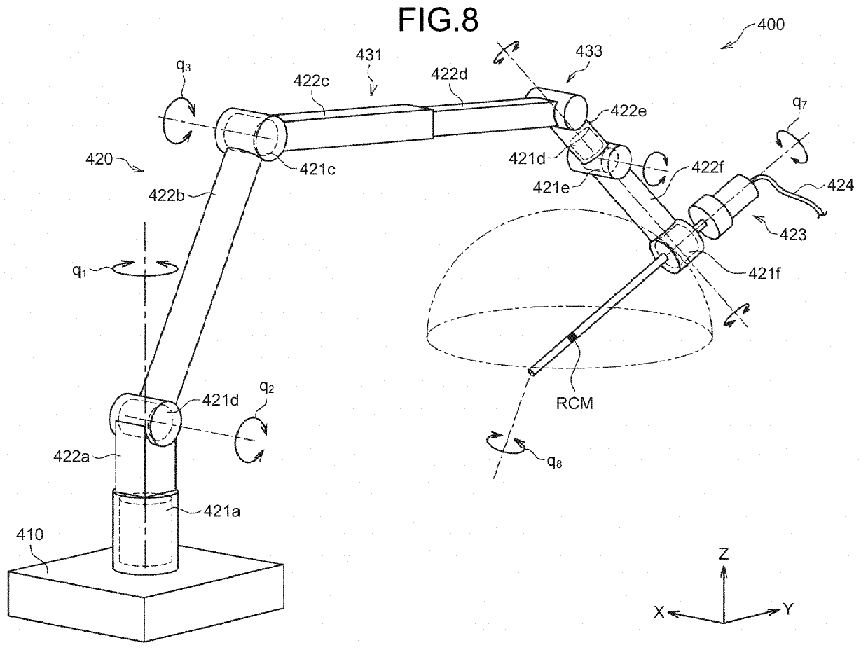

FIG. 8 is a schematic diagram illustrating an appearance of a support arm device 400 according to the present embodiment. The support arm device 400 corresponds to, for example, the robot arm A illustrated in FIGS. 1 to 5. Hereinafter, a schematic configuration of the support arm device 400 according to the present embodiment will be described with reference to FIG. 8.

The support arm device 400 according to the present embodiment includes a base portion 410 and an arm portion 420. The base portion 410 is a base of the support arm device 400, and the arm portion 420 extends from the base portion 410. Furthermore, although not illustrated in FIG. 8, a control unit that comprehensively controls the support arm device 400 may be provided in the base portion 410, and driving of the arm portion 420 may be controlled by the control unit. The control unit is implemented by, for example, various signal processing circuits such as a CPU and a digital signal processor (DSP).

The arm portion 420 includes a plurality of active joint portions 421a to 421f, a plurality of links 422a to 422f, and an endoscope device 423 as a distal end unit provided at a distal end of the arm portion 420.

The links 422a to 422f are substantially rod-shaped members. One end of the link 422a is connected to the base portion 410 via the active joint portion 421a, the other end of the link 422a is connected to one end of the link 422b via the active joint portion 421b, and the other end of the link 422b is connected to one end of the link 422c via the active joint portion 421c. The other end of the link 422c is connected to the link 422d via a passive slide mechanism 431, and the other end of the link 422d is connected to one end of the link 422e via a passive joint portion 433. The other end of the link 422e is connected to one end of the link 422f via the active joint portions 421d and 421e. The endoscope device 423 is connected to the distal end of the arm portion 420, that is, the other end of the link 422f via the active joint portion 421f. In this manner, the ends of the plurality of links 422a to 422f are connected to each other by the active joint portions 421a to 421f, the passive slide mechanism 431, and the passive joint portion 433 with the base portion 410 as a fulcrum, thereby forming an arm shape extending from the base portion 410.

A position and a posture of the endoscope device 423 are controlled by performing a control of driving actuators provided in the active joint portions 421a to 421f of the arm portion 420. In the present embodiment, a distal end of the endoscope device 423 enters the body cavity of the patient, which is the surgical site, and captures an image of a partial region of the surgical site. However, the distal end unit provided at the distal end of the arm portion 420 is not limited to the endoscope device 423, and various medical instruments may be connected to the distal end of the arm portion 420 as the distal end unit. As described above, the support arm device 400 according to the present embodiment is configured as a medical support arm device including a medical instrument.

Hereinafter, coordinate axes are defined as illustrated in FIG. 8 to describe the support arm device 400. Further, a top-bottom direction, a front-rear direction, and a left-right direction are defined in accordance with the coordinate axes. That is, a top-bottom direction with respect to the base portion 410 installed on a floor surface is defined as a z-axis direction and the top-bottom direction. Furthermore, a direction which is orthogonal to a z axis and in which the arm portion 420 extends from the base portion 410 (that is, a direction in which the endoscope device 423 is positioned with respect to the base portion 410) is defined as a y-axis direction and the front-rear direction. Further, a direction orthogonal to a y axis and the z axis are defined as an x-axis direction and the left-right direction.

The active joint portions 421a to 421f rotatably connect the links to each other. The active joint portions 421a to 421f each have actuators, and have a rotation mechanism that is rotated with respect to a predetermined rotation axis by driving of the actuators. It is possible to control the driving of the arm portion 420 such as extending or contracting (folding) of the arm portion 420 by controlling the rotation of each of the active joint portions 421a to 421f. Here, the driving of the active joint portions 421a to 421f can be controlled by a known whole body cooperative control and ideal joint control, for example. As described above, since the active joint portions 421a to 421f each have the rotation mechanism, in the following description, a driving control for the active joint portions 421a to 421f specifically means that rotation angles and/or generated torques (torques generated by the active joint portions 421a to 421f) of the active joint portions 421a to 421f are controlled.

The passive slide mechanism 431 is an aspect of a passive form change mechanism, and connects the link 422c and the link 422d to each other so as to be movable forward and backward along a predetermined direction. For example, the passive slide mechanism 431 may connect the link 422c and the link 422d to each other so as to be linearly movable. However, a forward and backward motion of the link 422c and the link 422d is not limited to a linear motion, and may be a forward and backward motion in a direction forming an arc shape. For example, the user moves the passive slide mechanism 100 forward and backward, such that a distance between the active joint portion 421c on one end side of the link 422c and the passive joint portion 433 varies. As a result, the overall form of the arm portion 420 can be changed.

The passive joint portion 433 is an aspect of the passive form change mechanism, and rotatably connects the link 422d and the link 422e to each other. For example, the user rotates the passive joint portion 433, such that an angle formed by the link 422d and the link 422e varies. As a result, the overall form of the arm portion 420 can be changed.

The support arm device 400 according to the present embodiment includes six active joint portions 421a to 421f, and six degrees of freedom are implemented when the arm portion 420 is driven. That is, while a driving control for the support arm device 400 is implemented by a driving control for the six active joint portions 421a to 421f by the control unit, the passive slide mechanism 431 and the passive joint portion 433 are not targets of a driving control performed by the control unit.

Specifically, as illustrated in FIG. 8, the active joint portions 421a, 421d, and 421f are provided so as to have, as rotation axis directions, a major axis direction of each of the connected links 422a and 422e and an imaging direction of the connected endoscope device 423. The active joint portions 421b, 421c, and 421e are provided so as to have, as the rotation axis direction, the x-axis direction which is a direction in which a connection angle of each of the connected links 422a to 422c, 422e, and 422f and the endoscope device 423 is changed in a y-z plane (a plane defined by the y axis and the z axis). As described above, in the present embodiment, the active joint portions 421a, 421d, and 421f have a function of performing so-called yawing, and the active joint portions 421b, 421c, and 421e have a function of performing so-called pitching.

With such a configuration of the arm portion 420, in the support arm device 400 according to the present embodiment, six degrees of freedom is implemented when the arm portion 420 is driven, and thus, the endoscope device 423 can be freely moved within a movable range of the arm portion 420. In FIG. 8, a hemisphere is illustrated as an example of the movable range of the endoscope device 423. Assuming that a central point of the hemisphere, remote center of motion (RCM), is the center of the image of the surgical site captured by the endoscope device 423, the image of the surgical site can be captured at various angles by moving the endoscope device 423 on a spherical surface of the hemisphere in a state in which the center of the image captured by the endoscope device 423 is fixed to the central point of the hemisphere.

The schematic configuration of the support arm device 400 according to the present embodiment has been described above. Next, the whole body cooperative control and the ideal joint control for controlling the driving of the arm portion 420 in the support arm device 400 according to the present embodiment, that is, the driving of the active joint portions 421a to 421f, will be described.

Note that, although a case where the arm portion 220 of the support arm device 400 has a plurality of joint portions and has six degrees of freedom has been described, the present disclosure is not limited thereto. Specifically, the arm portion 220 may have a structure in which the endoscope device 423 or an exoscope is provided at the distal end. For example, the arm portion 220 may have a configuration having only one degree of freedom with which the endoscope device 423 is driven to move in a direction in which the endoscope device enters the body cavity of the patient and a direction in which the endoscope device moves backward.

<2-3. Specific Configuration Example of Endoscope>

An endoscope can be installed in the support arm device of the present embodiment. Hereinafter, a basic configuration of an oblique-viewing endoscope will be described as an example of the endoscope of the present embodiment. Note that the endoscope of the present embodiment is not limited to the oblique-viewing endoscope described below.

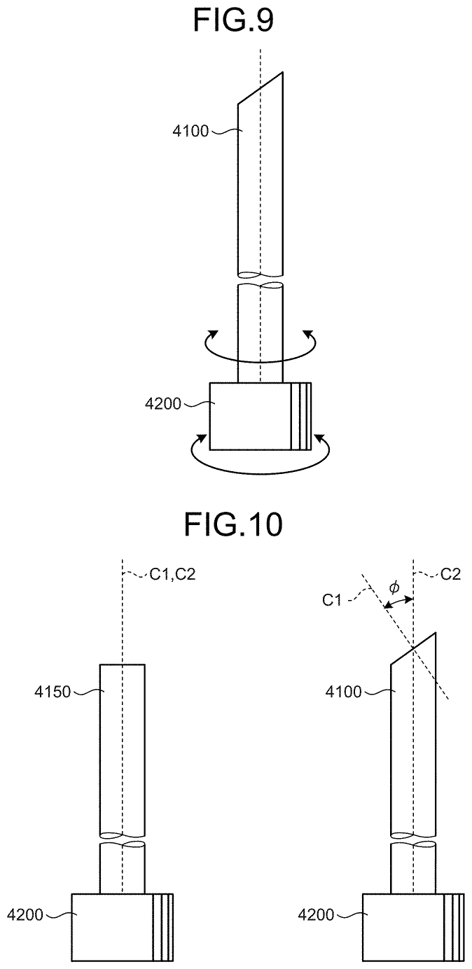

FIG. 9 is a schematic diagram illustrating a configuration of an oblique-viewing endoscope 4100 according to an embodiment of the present disclosure. As illustrated in FIG. 9, the oblique-viewing endoscope 4100 is attached to a distal end of a camera head 4200. The oblique-viewing endoscope 4100 corresponds to the lens barrel 5003 described with reference to FIGS. 6 and 7, and the camera head 4200 corresponds to the camera head 5005 described with reference to FIGS. 6 and 7. The oblique-viewing endoscope 4100 and the camera head 4200 are rotatable independently of each other. An actuator is provided between the oblique-viewing endoscope 4100 and the camera head 4200 similarly to each of the joint portions 5033a, 5033b, and 5033c, and the oblique-viewing endoscope 4100 rotates with respect to the camera head 4200 by driving of the actuator.

The oblique-viewing endoscope 4100 is supported by the support arm device 5027. The support arm device 5027 has a function of holding the oblique-viewing endoscope 4100 instead of the scopist and moving the oblique-viewing endoscope 4100 so that a desired site can be observed according to an operation performed by the operator or the assistant.

Note that the endoscope of the present embodiment is not limited to an oblique-viewing endoscope. The endoscope of the present embodiment may be a forward-viewing endoscope. FIG. 10 is a schematic view illustrating the oblique-viewing endoscope 4100 and a forward-viewing endoscope 4150 in comparison. In the forward-viewing endoscope 4150, an orientation (C1) of the objective lens toward a subject coincides with a longitudinal direction (C2) of the forward-viewing endoscope 4150. On the other hand, in the oblique-viewing endoscope 4100, a predetermined angle ϕ is formed between the orientation (C1) of the objective lens toward the subject and the longitudinal direction (C2) of the oblique-viewing endoscope 4100. Note that in a case where the angle ϕ is 90 degrees, the oblique-viewing endoscope 4100 is called a side-viewing endoscope. It is a matter of course that the endoscope of the present embodiment may be a side-viewing endoscope.

<2-4. Second Configuration Example (Medical Observation System)>

Next, a configuration of a medical observation system 1 will be described as another configuration example of the medical system of the present embodiment. Note that the support arm device 400 and the oblique-viewing endoscope 4100 described above can also be applied to the medical observation system described below. In addition, the medical observation system described below may be regarded as a functional configuration example or a modification of the endoscopic surgery system 5000 described above.

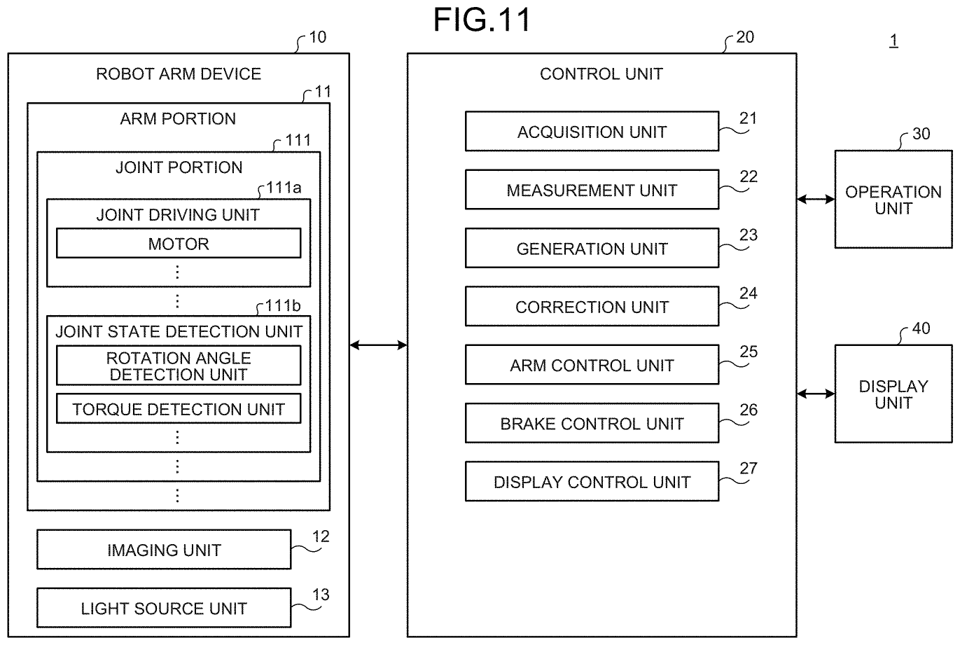

FIG. 11 is a block diagram illustrating an example of a configuration of the medical observation system 1 according to an embodiment of the present disclosure. Hereinafter, a configuration of the medical observation system according to the embodiment of the present disclosure will be described with reference to FIG. 11.

As illustrated in FIG. 11, the medical observation system 1 includes a robot arm device 10, a control unit 20, an operation unit 30, and a display unit 40.

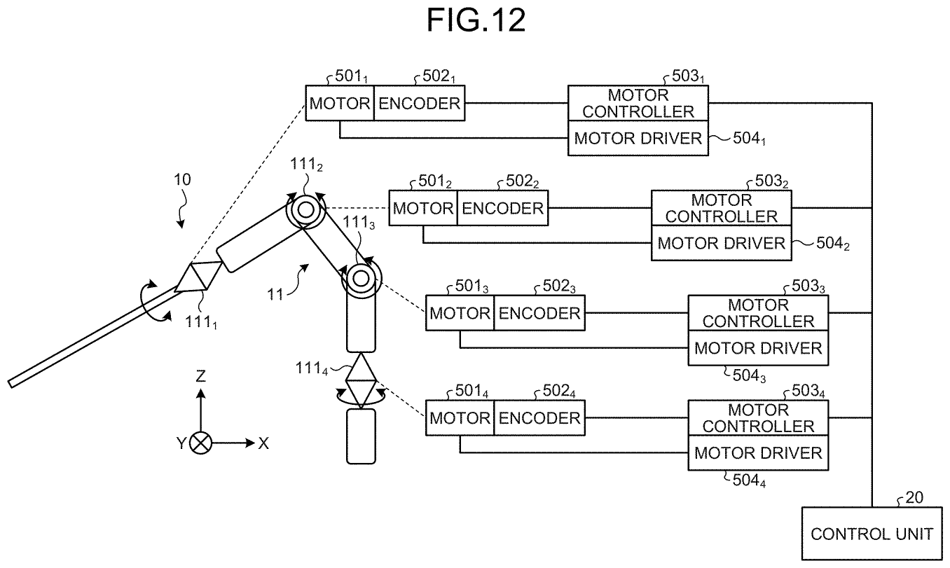

FIG. 12 is a diagram illustrating a specific configuration example of the robot arm device 10 according to the embodiment of the present disclosure. The robot arm device 10 includes, for example, an arm portion 11 (articulated arm) that is a multilink structure including a plurality of joint portions and a plurality of links. The robot arm device 10 corresponds to, for example, the robot arm A illustrated in FIGS. 1 to 5 or the support arm device 400 illustrated in FIG. 8. The robot arm device 10 is operated under the control of the control unit 20. The robot arm device 10 controls a position and a posture of a distal end unit (for example, an endoscope) provided at a distal end of the arm portion 11 by driving the arm portion 11 within a movable range. The arm portion 11 corresponds to, for example, the arm portion 420 illustrated in FIG. 8.

The arm portion 11 includes a plurality of joint portions 111. FIG. 11 illustrates a configuration of one joint portion 111 as a representative of the plurality of joint portions.

The joint portion 111 rotatably connects the links in the arm portion 11, and rotation thereof is controlled under the control of the control unit 20, thereby driving the arm portion 11. The joint portions 111 correspond to, for example, the active joint portions 421a to 421f illustrated in FIG. 8. Furthermore, the joint portion 111 may have an actuator.

As illustrated in FIG. 11, the joint portion 111 includes one or more joint driving units 111a and one or more joint state detection units 111b.

The joint driving unit 111a is a driving mechanism in the actuator of the joint portion 111, and the joint driving unit 111a performs driving to rotate the joint portion 111. The joint driving unit 111a corresponds to a motor 501₁ illustrated in FIG. 12 and the like. The driving of the joint driving unit 111a is controlled by an arm control unit 25. For example, the joint driving unit 111a corresponds to a motor and a motor driver. The driving performed by the joint driving unit 111a corresponds to, for example, driving the motor by the motor driver with a current amount according to a command from the control unit 20.

The joint state detection unit 111b is, for example, a sensor that detects a state of the joint portion 111. Here, the state of the joint portion 111 may mean a state of a motion of the joint portion 111. For example, the state of the joint portion 111 includes information such as a rotation angle, a rotation angular speed, a rotation angular acceleration, and a generated torque of the joint portion 111. The joint state detection unit 111b corresponds to an encoder 502₁ and the like illustrated in FIG. 12.

In the present embodiment, the joint state detection unit 111b functions as, for example, a rotation angle detection unit that detects the rotation angle of the joint portion 111 and a torque detection unit that detects the generated torque of the joint portion 111 and an external torque. Note that the rotation angle detection unit and the torque detection unit may be an encoder and a torque sensor of the actuator, respectively. The joint state detection unit 111b transmits the detected state of the joint portion 111 to the control unit 20. Note that the joint state detection unit 111b can also be regarded as a measurement unit that measures a load applied to the motor. Here, the load is, for example, a load torque.

Returning to FIG. 11, the imaging unit 12 is provided at the distal end of the arm portion 11 and captures images of various imaging targets. The imaging unit 12 captures, for example, an operative field image including various medical instruments, organs, and the like in the abdominal cavity of the patient. Specifically, the imaging unit 12 is a camera or the like capable of capturing an image of the imaging target in a form of a moving image or a still image. More specifically, the imaging unit 12 is a wide-angle camera including a wide-angle optical system. That is, the operative field image is an operative field image captured by the wide-angle camera. For example, although an angle of view of a normal endoscope is about 80°, an angle of view of the imaging unit 12 according to the present embodiment may be 140°. Note that the angle of view of the imaging unit 12 may be greater than 80° and less than 140°, or may be equal to or greater than 140°. The imaging unit 12 transmits an electric signal (image signal) corresponding to the captured image to the control unit 20. Note that, in FIG. 11, the imaging unit 12 does not need to be included in the robot arm device, and an aspect thereof is not limited as long as the imaging unit 12 is supported by the arm portion 11.

In the light source unit 13, the imaging unit 12 irradiates the imaging target with light. The light source unit 13 can be implemented by, for example, a wide-angle lens LED. For example, the light source unit 13 may be implemented by combining a normal LED and a lens to diffuse light. In addition, the light source unit 13 may be configured to diffuse (increase the angle of) light transmitted through an optical fiber with a lens. Further, the light source unit 13 may expand an irradiation range by irradiating the optical fiber itself with light in a plurality of directions. Note that, in FIG. 6, the light source unit 13 does not need to be included in the robot arm device 10, and an aspect thereof is not limited as long as the irradiation light can be guided to the imaging unit 12 supported by the arm portion 11.

Hereinafter, a specific configuration example of the robot arm device 10 according to the embodiment of the present disclosure will be described with reference to FIG. 12.

For example, as illustrated in FIG. 12, the arm portion 11 of the robot arm device 10 includes a first joint portion 111₁, a second joint portion 111₂, a third joint portion 111₃, and a fourth joint portion 111₄.

The first joint portion 111₁ includes the motor 501₁, the encoder 502₁, a motor controller 503₁, and a motor driver 504₁. Since the second joint portion 111₂ to the fourth joint portion 111₄ also have the same configuration as the first joint portion 111₁, the first joint portion 111₁ will be described below as an example.

Note that each of the joint portions including the first joint portion 111₁ may include a b5rake of the motor 501. At this time, the brake may be a mechanical brake. Then, the joint portion may be configured to maintain a current state of the arm portion 11 by using the brake, for example, in a case where the motor is not operated. Even in a case where supply of power to the motor is stopped for some reason, since the arm portion 11 is fixed by the mechanical brake, the endoscope does not move to an unintended position.

The motor 501₁ is driven under the control of the motor driver 504₁ to drive the first joint portion 111₁. The motor 501₁ and/or the motor driver 504₁ corresponds to, for example, the joint driving unit 111a illustrated in FIG. 11. The motor 501₁ drives the first joint portion 111₁ in a direction of an arrow attached to the first joint portion 111₁, for example. The motor 501₁ controls the position and the posture of the arm portion 11 or positions and postures of the lens barrel and the camera by driving the first joint portion 111₁. Note that, in the present embodiment, as one form of the endoscope, a camera (for example, the imaging unit 12) may be provided at a distal end of a lens barrel.

The encoder 502₁ detects information regarding a rotation angle of the first joint portion 111₁ under the control of the motor controller 503₁. That is, the encoder 502₁ acquires information regarding the posture of the first joint portion 111₁. The encoder 502₁ detects information regarding a torque of the motor under the control of the motor controller 5031.

The control unit 20 controls the position and the posture of the arm portion 11. Specifically, the control unit 20 controls the motor controllers 5031 to 5034, the motor drivers 5041 to 5044, and the like to control the first joint portion 1111 to the fourth joint portion 1114. By doing so, the control unit 20 controls the position and the posture of the arm portion 11. The control unit 20 may be included in the robot arm device 10 or may be a device separate from the robot arm device 10. The control unit 20 corresponds to, for example, the control device that controls the robot arm A illustrated in FIGS. 1 to 5. Alternatively, the control unit 20 corresponds to, for example, the CCU 5039 or the arm control device 5045 illustrated in FIG. 6.

The control unit 20 is implemented by, for example, a central processing unit (CPU), a micro processing unit (MPU), or the like executing a program (for example, a program according to the present invention) stored in a storage unit (not illustrated) with a random access memory (RAM) or the like as a work area. Further, the control unit 20 is a controller and may be implemented by, for example, an integrated circuit such as an application specific integrated circuit (ASIC) or a field programmable gate array (FPGA).

As illustrated in FIG. 11, the control unit 20 includes an acquisition unit 21, a measurement unit 22, a generation unit 23, a correction unit 24, an arm control unit 25, a brake control unit 26, and a display control unit 27. The respective blocks (the acquisition unit 21, the display control unit 27, and the like) included in the control unit 20 are functional blocks each indicating the function of the control unit 20. These functional blocks may be software blocks or hardware blocks. For example, each of the above-described functional blocks may be one software module implemented by software (including a microprogram) or may be one circuit block on a semiconductor chip (die). It is a matter of course that each functional block may be one processor or one integrated circuit. A method of configuring the functional block is arbitrary. Note that the control unit 20 may be configured with a functional unit different from the above-described functional block.

For example, the acquisition unit 21 acquires an instruction from a user (for example, the operator or a person assisting the operator) who operates the operation unit 30. For example, the acquisition unit 21 acquires an instruction regarding backward movement of the endoscope.

For example, the measurement unit 22 measures a load (for example, load torque) applied to a motor that drives the joint 111 of the arm portion 11. For example, the measurement unit 22 acquires information regarding the torque of the motor installed in the joint portion 111 from a sensor (for example, the joint state detection unit 111b that functions as a torque sensor) installed in the joint portion 111, and measures the load applied to the motor on the basis of the acquired information. Note that a plurality of motors can be installed in the arm portion 11. For example, it is assumed that one motor is installed in each joint portion in a case where the arm portion 11 includes a plurality of joint portions 111. It is a matter of course that a case where a plurality of motors are installed in one joint can also be assumed. In a case where a plurality of motors are installed in the arm portion 11, the number of motors whose load is measured by the measurement unit 22 may be one or plural.

As described above, while the motor is not operated, the arm portion 11 is fixed to maintain the current position by the mechanical brake. Therefore, for example, the measurement unit 22 may cause the brake control unit 26 to temporarily release the brake and measure the load on the motor during the temporary release. Since the brake is temporarily released, the endoscope does not move to an unintended position even in a case where supply of power to the motor is stopped for some reason. Furthermore, since the load is measured while the brake is released, the load applied to the motor due to movement of the trocar can also be accurately detected.

The generation unit 23 generates three-dimensional information in the body into which the endoscope is inserted. For example, the generation unit 23 generates the three-dimensional information on the basis of sensing information in the body. For example, the generation unit 23 generates the three-dimensional information on the basis of information regarding the inside of the body acquired by a distance measuring sensor that measures a distance inside the body. Furthermore, for example, the generation unit 23 generates the three-dimensional information on the basis of information regarding the inside of the body acquired by one or more cameras for capturing an image of the inside of the body. Furthermore, for example, the generation unit 23 generates the three-dimensional information on the basis of information regarding the inside of the body acquired by the endoscope. Note that the three-dimensional information may be the in-vivo environment map generated by simultaneous localization and mapping (SLAM).

The correction unit 24 corrects the three-dimensional information on the basis of the load of the motor measured by the measurement unit 22. For example, the correction unit 24 corrects the three-dimensional information on the basis of information regarding a change in measured load. At this time, the correction unit 24 may correct the three-dimensional information on the basis of an increase or decrease in measured load. For example, the correction unit 24 may correct the three-dimensional information in an approaching direction in a case where the measured load is increased, and may correct the three-dimensional information in a separating direction in a case where the measured load is decreased. Note that the correction unit 24 may correct the three-dimensional information on the basis of the load measured when the brake is released.

The arm control unit 25 comprehensively controls the robot arm device 10 and controls the driving of the arm portion 11. Specifically, the arm control unit 25 controls the driving of the arm portion 11 by controlling the driving of the joint portion 111. More specifically, the arm control unit 25 controls a rotation speed of the motor by controlling the amount of current supplied to the motor in the actuator of the joint portion 111, thereby controlling the rotation angle and the generated torque of the joint portion 111.

The arm control unit 25 controls the operation of the motor that drives the arm on the basis of the corrected three-dimensional information. For example, the arm control unit 25 recognizes the surgical instrument inserted into the body from a video captured by the endoscope, and controls the operation of the motor so that imaging is performed by the endoscope at a predetermined position (for example, an area of interest of the operator) on the basis of the corrected three-dimensional information and a result of recognizing the surgical instrument. Note that the arm control unit 25 may move the endoscope toward the outside of the body on the basis of the corrected three-dimensional information in a case where the acquisition unit 21 acquires the instruction regarding backward movement of the endoscope from the user. For example, the arm control unit 25 may move the endoscope inserted into the body through the trocar to the position of the trocar in a case where the acquisition unit 21 acquires the instruction regarding backward movement of the endoscope.

The brake control unit 26 controls the brake of the motor that drives the arm. As described above, the measurement unit 22 measures the load on the motor while the brake is temporarily released by the brake control unit 26. A brake release time may be, for example, 0.1 seconds to 1 second, which is short. It is a matter of course that the brake release time is not limited to 0.1 seconds to 1 second.

The display control unit 27 causes the display unit 40 to display various images (including not only still images but also videos). For example, the display control unit 27 causes the display unit 40 to display the image captured by the imaging unit 12.

The operation unit 30 receives various types of operation information from the user. The operation unit 30 is implemented by, for example, a microphone that detects a voice, a gaze sensor that detects a gaze, a switch that receives a physical operation, or a touch panel. The operation unit 30 may be implemented by other physical mechanisms.

The display unit 40 displays various images. The display unit 40 is, for example, a display. For example, the display unit 40 may be a display such as a liquid crystal display (LCD) or an organic electroluminescence (EL) display. For example, the display unit 40 displays an image captured by the imaging unit 12.

<<3. Operation of Medical System>>

The configuration of the medical system has been described above, and the operation of the medical system will be described below. In the following description, an example in which a support arm that supports an endoscope is controlled will be described.

Note that although it is assumed in the following description that the medical system of the present embodiment is the medical observation system 1, the operation described below can be applied not only to the medical observation system 1 but also to other medical systems.

<3-1. Example of Control of Arm (Control Based on SLAM)>

First, an example in which the robot arm A (for example, the robot arm device 10 or the support arm device 400) is controlled on the basis of SLAM will be described.

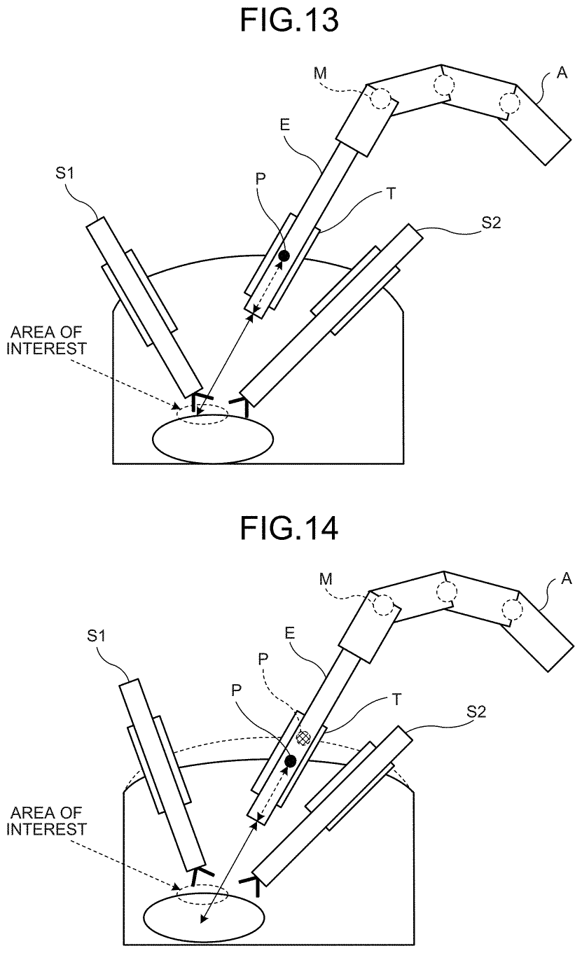

In the present control example, the control device of the robot arm A aligns the endoscope E with the area of interest of the operator on the basis of the in-vivo environment map created by SLAM. FIGS. 13 and 14 are diagrams illustrating a state in which the endoscope E captures an image of the area of interest.

FIG. 13 illustrates a state in which the endoscope E is held in the area of interest of the operator on the basis of the information of the in-vivo map after the in-vivo map is created in a state in which the pneumoperitoneum is sufficient. In the example of FIG. 13, since the endoscope E is positioned at an appropriate distance from the area of interest, the image displayed on the display unit has an expected size.

On the other hand, FIG. 14 illustrates a state in which the pneumoperitoneum is insufficient. Since the control device of the robot arm A aligns the position of the endoscope E on the basis of coordinates of the trocar point P in a state in which the pneumoperitoneum is sufficient, when the carbon dioxide gas escapes from the body and the pneumoperitoneum is changed, the position of the endoscope E becomes closer to the area of interest than expected. Therefore, the image displayed on the display unit is an image enlarged more than expected.

Therefore, the control device of the robot arm A corrects the in-vivo map (three-dimensional information) on the basis of the load of the motor that drives the arm. Note that, in the following description, the control unit 20 of the robot arm device 10 performs the following processing, but the control device that performs the following processing is not limited to the control unit 20. For example, the control device that performs the following processing may be the control device of the robot arm A, or may be the CCU 5039 or the arm control device 5045 illustrated in FIG. 6.

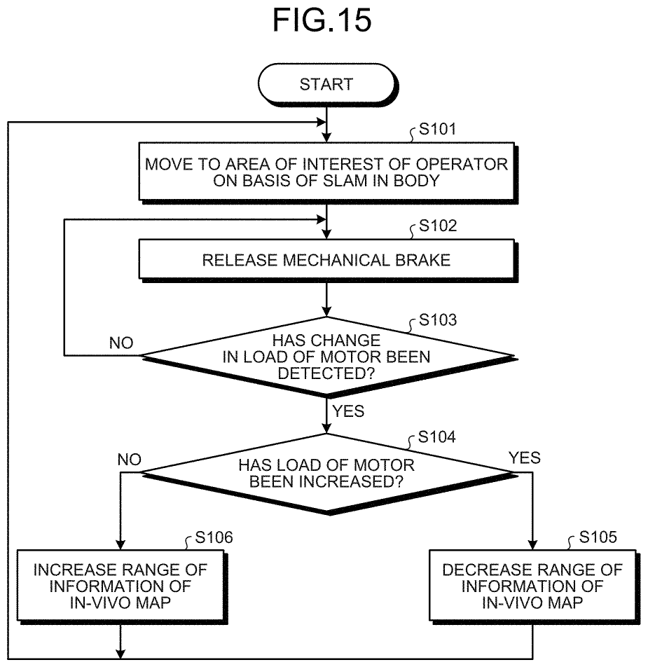

Hereinafter, control processing according to an embodiment of the present invention will be described with reference to FIG. 15. FIG. 15 is a flowchart illustrating an example of control processing for holding the endoscope in the area of interest of the operator. In the following description, it is assumed that the generation unit 23 generates the in-vivo map by SLAM. Further, it is assumed that the brake control unit 26 brakes the motor to hold the arm portion 11 at a fixed position.

First, the control unit 20 moves the endoscope to the area of interest of the operator on the basis of SLAM (Step S101). At this time, the control unit 20 may control the robot arm device 10 by using the information of the in-vivo map corrected in Step S105 or Step S106 described later. Thereafter, the control unit 20 periodically releases the mechanical brake in order to enable measurement of the load applied to the motor (Step S102).

Then, the control unit 20 detects whether or not there is a change in load of the motor due to a change in trocar position (Step S103). In a case where there is no change in load (Step S103: No), the control unit 20 returns the processing to Step S102.

In a case where there is a change in load (Step S103: Yes), the control unit 20 determines whether the load has been increased or decreased (Step S104).

In a case where the load of the motor has been increased (Step S104: Yes), the control unit 20 determines that the trocar descends and performs correction to decrease a range of the information of the in-vivo map created by SLAM in the body (Step S105).

On the other hand, in a case where the load of the motor has been decreased (Step S104: No), the control unit 20 determines that the trocar ascends and performs correction to increase the range of the information of the in-vivo map (Step S106).

Then, the control unit 20 returns to Step S101, and controls the operation of the motor that drives the arm on the basis of the corrected information of the in-vivo map.

As a result, the robot arm device 10 can be controlled on the basis of the information of the in-vivo map with high accuracy, such that the control unit 20 can hold the endoscope at a position suitable for surgery.

<3-2. Example of Control of Arm (Control in Consideration of Tracking of Surgical Instrument)>

Next, an example in which a control is performed in consideration of tracking of the surgical instrument will be described.

In the present control example, the control device of the robot arm A tracks the surgical instrument during surgery. In the following description, similarly to the above-described control example, it is assumed that the control unit 20 of the robot arm device 10 performs the following processing. It is a matter of course that the control device that performs the following processing is not limited to the control unit 20.

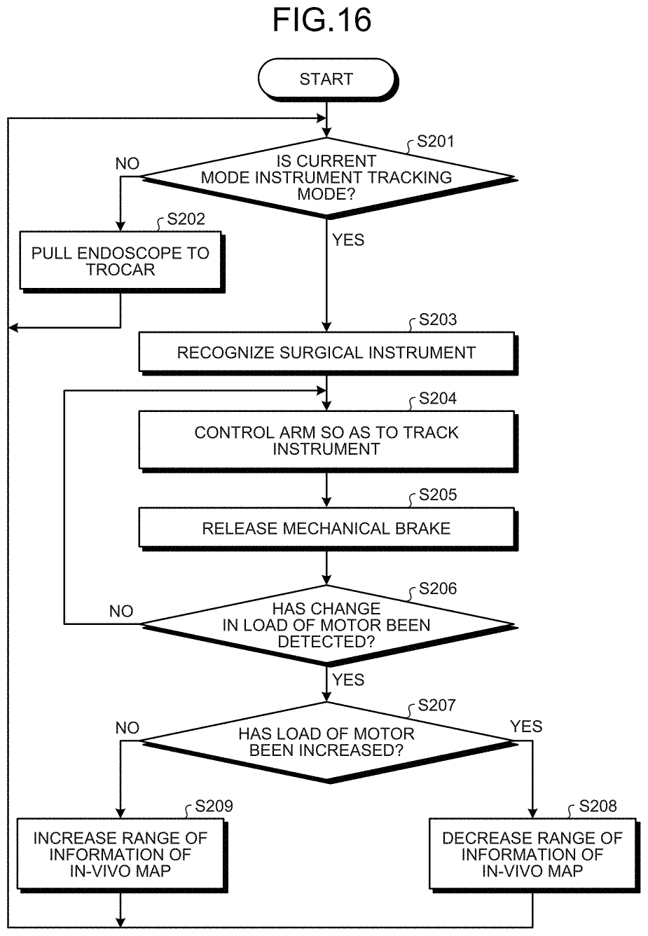

Hereinafter, control processing according to an embodiment of the present invention will be described with reference to FIG. 16. FIG. 16 is a flowchart illustrating an example of processing for controlling the position of the endoscope so as to track the surgical instrument during surgery. In the following description, it is assumed that the generation unit 23 generates the in-vivo map by SLAM. Further, it is assumed that the brake control unit 26 brakes the motor to hold the arm portion 11 at a fixed position.

First, the control unit 20 determines whether a current mode is a surgical instrument tracking mode or a surgical instrument insertion preparation mode (Step S201). The surgical instrument tracking mode is a mode in which the position of the endoscope is controlled so as to track the surgical instrument. In addition, the surgical instrument insertion preparation mode is a mode in which a space for inserting the instrument is secured by pulling the endoscope to the trocar in order to insert the instrument into the body. Note that information used by the control unit 20 to determine the mode may be information acquired from the user via the operation unit 30.

In a case where the current mode is the surgical instrument insertion preparation mode (Step S201: No), the control unit 20 pulls the endoscope to the trocar on the basis of the information of the in-vivo map to secure the space for inserting the instrument (Step S202). At this time, the control unit 20 may control the robot arm device 10 by using the information of the in-vivo map corrected in Step S208 or Step S209 described later.

On the other hand, in a case where the current mode is the surgical instrument tracking mode (Step S201: Yes), the control unit 20 recognizes the surgical instrument from the image captured by the endoscope (Step S203). Once the instrument is recognized, the control unit 20 controls the motor that drives the arm portion 11 so as to track the instrument (Step S204). At this time, the control unit 20 may control the robot arm device 10 by using the information of the in-vivo map corrected in Step S208 or Step S209 described later.

Thereafter, once the instrument is stabilized at a certain position, the control unit 20 brakes the motor to hold the arm portion 11 at the certain position. Then, the control unit 20 periodically releases the mechanical brake in order to enable measurement of the load applied to the motor (Step S205).

Then, the control unit 20 detects whether or not there is a change in load of the motor due to a change in trocar position (Step S206). In a case where there is no change in load (Step S206: No), the control unit 20 returns the processing to Step S204.

In a case where there is a change in load (Step S206: Yes), the control unit 20 determines whether the load has been increased or decreased (Step S207).

In a case where the load of the motor has been increased (Step S207: Yes), the control unit 20 determines that the trocar descends and performs correction to decrease a range of the information of the in-vivo map created by SLAM in the body (Step S208).

On the other hand, in a case where the load of the motor has been decreased (Step S207: No), the control unit 20 determines that the trocar ascends and performs correction to increase the range of the information of the in-vivo map (Step S209).

Then, the control unit 20 returns to Step S201, and controls the operation of the motor that drives the arm on the basis of the corrected information of the in-vivo map.

As a result, the robot arm device 10 can be controlled on the basis of the information of the in-vivo map with high accuracy, such that the control unit 20 can accurately track the surgical instrument. In addition, since the robot arm device 10 can be controlled on the basis of the information of the in-vivo map with high accuracy, the control unit 20 can accurately pull the endoscope to the trocar.

<<4. Modification>>

The control device (for example, the control device of the robot arm A, the CCU 5039, the arm control device 5045, or the control unit 20) that controls the support arm of the present embodiment may be implemented by a dedicated computer system or a general-purpose computer system.

For example, a program for performing the above-described control processing is stored in a computer-readable recording medium such as an optical disk, a semiconductor memory, a magnetic tape, or a flexible disk, and distributed. Then, for example, the control device is implemented by installing the program in a computer and performing the above processing. At this time, the control device may be a device (for example, a personal computer) outside the support arm (for example, a medical support arm such as the robot arm A, the support arm device 5027, the support arm device 400, or the robot arm device 10). Furthermore, the control device may be a device (for example, a processor mounted on the support arm) inside the support arm.

Further, the communication program may be stored in a disk device included in a server device on a network such as the Internet, and be downloaded to a computer. Further, the functions described above may be implemented by cooperation between an operating system (OS) and application software. In this case, the part other than the OS may be stored in a medium and distributed, or the part other than the OS may be stored in the server device and downloaded to a computer.

Further, among the respective processing described in the above-described embodiments, all or some of the processing described as being automatically performed can be manually performed. Alternatively, all or some of the processing described as being manually performed can be automatically performed by a known method. In addition, the processing procedures, specific names, information including various data and parameters illustrated in the specification and drawings can be arbitrarily changed unless otherwise specified. For example, various information illustrated in each drawing is not limited to the illustrated information.

Further, each illustrated component of each device is functionally conceptual, and does not necessarily have to be configured physically as illustrated in the drawings. That is, the specific modes of distribution/integration of the respective devices are not limited to those illustrated in the drawings. All or some of the devices can be functionally or physically distributed/integrated in any arbitrary unit, depending on various loads or the status of use.

Further, the above-described embodiments can be appropriately combined as long as the processing contents do not contradict each other. Further, the order of each step illustrated in the flowchart of the above-described embodiment can be changed as appropriate.

Furthermore, for example, the present embodiment can be implemented as any component included in the device or system, such as a processor as a system large scale integration (LSI) or the like, a module using a plurality of processors or the like, a unit using a plurality of modules or the like, a set obtained by further adding other functions to a unit, or the like (that is, some components of the device).

Note that, in the present embodiment, the system means a set of a plurality of components (devices, modules (parts), and the like), and it does not matter whether or not all the components are in the same housing. Therefore, a plurality of devices housed in separate housings and connected via a network and one device in which a plurality of modules are housed in one housing are both systems.

Furthermore, for example, the present embodiment can adopt a configuration of cloud computing in which one function is shared and processed by a plurality of devices in cooperation via a network.

<<5. Conclusion>>

The medical support arm of the present embodiment includes the support arm that supports the endoscope, and the motor that drives the support arm. The control device that controls the support arm generates the three-dimensional information in the body into which the endoscope is inserted, and measures the load applied to the motor. The control device corrects the three-dimensional information on the basis of the measured load.

As a result, the instruction arm can be controlled on the basis of highly accurate three-dimensional information, such that the medical support arm can hold the endoscope at a position suitable for surgery.

Note that the effects described in the present specification are merely examples. The effects of the present disclosure are not limited thereto, and other effects may be obtained.

Note that the present technology can also have the following configurations.
(1)

A medical support arm comprising:

a support arm that supports an endoscope;

an actuator that drives the support arm;

a measurement unit that measures a load applied to the actuator;

a generation unit that generates three-dimensional information in a body into which the endoscope is inserted; and

a correction unit that corrects the three-dimensional information on a basis of the measured load.
(2)

The medical support arm according to (1), further comprising an arm control unit that controls an operation of the actuator on a basis of the corrected three-dimensional information.
(3)

The medical support arm according to (2), wherein the arm control unit recognizes a surgical instrument inserted into the body from a video captured by the endoscope, and controls the operation of the actuator so that imaging is performed by the endoscope at a predetermined position on a basis of the corrected three-dimensional information and a result of recognizing the surgical instrument.
(4)

The medical support arm according to (2) or (3), further comprising an acquisition unit that acquires an instruction regarding backward movement of the endoscope,

wherein the arm control unit moves the endoscope toward an outside of the body on a basis of the corrected three-dimensional information in a case where the instruction is acquired.
(5)

The medical support arm according to (4), wherein the arm control unit moves the endoscope inserted into the body through a trocar to a position of the trocar in a case where the instruction is acquired.
(6)

The medical support arm according to any one of (1) to (5), further comprising a brake control unit that controls a brake of the actuator,

wherein the brake control unit periodically releases the brake, and

the correction unit corrects the three-dimensional information on a basis of the load measured when the brake is released.
(7)

The medical support arm according to any one of (1) to (6), wherein the correction unit corrects the three-dimensional information on a basis of information regarding a change in measured load.
(8)

The medical support arm according to (7), wherein the correction unit corrects the three-dimensional information on a basis of an increase or decrease in measured load.
(9)

The medical support arm according to (8), wherein the correction unit performs correction so as to decrease a range of the three-dimensional information in a case where the measured load has been increased, and performs correction so as to increase the range of the three-dimensional information in a case where the measured load has been decreased.
(10)

The medical support arm according to any one of (1) to (9), wherein the generation unit generates the three-dimensional information on a basis of information regarding an inside of the body acquired by the endoscope.
(11)

The medical support arm according to (10), wherein the generation unit generates the three-dimensional information on a basis of information regarding the inside of the body acquired by a distance measuring sensor that measures a distance in the body.
(12)

The medical support arm according to (10), wherein the generation unit generates the three-dimensional information on a basis of information regarding the inside of the body acquired by one or more cameras that capture an image of the inside of the body.
(13)

The medical support arm according to any one of (1) to (12), wherein the three-dimensional information is an in-vivo map generated by simultaneous localization and mapping (LAM).
(14)

A medical system comprising:

a support arm that supports an endoscope; and

an arm control device that controls the support arm,

wherein the support arm includes an actuator that drives the support arm, and a sensor that measures a load applied to the actuator, and

the arm control device includes a generation unit that generates three-dimensional information in a body into which the endoscope is inserted, and a correction unit that corrects the three-dimensional information on a basis of the measured load.
(15)

A control device including:

a measurement unit that measures a load applied to an actuator that drives a support arm supporting an endoscope;

a generation unit that generates three-dimensional information in a body into which the endoscope is inserted; and

a correction unit that corrects the three-dimensional information on the basis of the measured load.
(16)

A control method including:

measuring a load applied to an actuator that drives a support arm supporting an endoscope;

generating three-dimensional information in a body into which the endoscope is inserted; and

correcting the three-dimensional information on the basis of the measured load.
(17)

A program for causing a computer to function as:

a measurement unit that measures a load applied to an actuator that drives a support arm supporting an endoscope;

a generation unit that generates three-dimensional information in a body into which the endoscope is inserted; and

a correction unit that corrects the three-dimensional information on the basis of the measured load.

### REFERENCE SIGNS LIST

1 MEDICAL OBSERVATION SYSTEM

10 ROBOT ARM DEVICE

11 ARM PORTION

111 JOINT PORTION

111a JOINT DRIVING UNIT

111b JOINT STATE DETECTION UNIT

12 IMAGING UNIT

13 LIGHT SOURCE UNIT

20 CONTROL UNIT

21 ACQUISITION UNIT

22 MEASUREMENT UNIT

23 GENERATION UNIT

24 CORRECTION UNIT

25 ARM CONTROL UNIT

26 BRAKE CONTROL UNIT

27 DISPLAY CONTROL UNIT

30 OPERATION UNIT

40 DISPLAY UNIT

1. A medical support arm comprising:
a support arm that supports an endoscope;
an actuator that drives the support arm;
a measurement unit that measures a load applied to the actuator;
a generation unit that generates three-dimensional information in a body into which the endoscope is inserted; and
a correction unit that corrects the three-dimensional information on a basis of the measured load.

2. The medical support arm according to claim 1, further comprising an arm control unit that controls an operation of the actuator on a basis of the corrected three-dimensional information.

3. The medical support arm according to claim 2, wherein the arm control unit recognizes a surgical instrument inserted into the body from a video captured by the endoscope, and controls the operation of the actuator so that imaging is performed by the endoscope at a predetermined position on a basis of the corrected three-dimensional information and a result of recognizing the surgical instrument.

4. The medical support arm according to claim 2, further comprising an acquisition unit that acquires an instruction regarding backward movement of the endoscope,
wherein the arm control unit moves the endoscope toward an outside of the body on a basis of the corrected three-dimensional information in a case where the instruction is acquired.

5. The medical support arm according to claim 4, wherein the arm control unit moves the endoscope inserted into the body through a trocar to a position of the trocar in a case where the instruction is acquired.

6. The medical support arm according to claim 1, further comprising a brake control unit that controls a brake of the actuator,
wherein the brake control unit periodically releases the brake, and
the correction unit corrects the three-dimensional information on a basis of the load measured when the brake is released.

7. The medical support arm according to claim 1, wherein the correction unit corrects the three-dimensional information on a basis of information regarding a change in measured load.

8. The medical support arm according to claim 7, wherein the correction unit corrects the three-dimensional information on a basis of an increase or decrease in measured load.

9. The medical support arm according to claim 8, wherein the correction unit performs correction so as to decrease a range of the three-dimensional information in a case where the measured load has been increased, and performs correction so as to increase the range of the three-dimensional information in a case where the measured load has been decreased.

10. The medical support arm according to claim 1, wherein the generation unit generates the three-dimensional information on a basis of information regarding an inside of the body acquired by the endoscope.

11. The medical support arm according to claim 10, wherein the generation unit generates the three-dimensional information on a basis of information regarding the inside of the body acquired by a distance measuring sensor that measures a distance in the body.

12. The medical support arm according to claim 10, wherein the generation unit generates the three-dimensional information on a basis of information regarding the inside of the body acquired by one or more cameras that capture an image of the inside of the body.

13. The medical support arm according to claim 1, wherein the three-dimensional information is an in-vivo map generated by simultaneous localization and mapping (SLAM).

14. A medical system comprising:
a support arm that supports an endoscope; and
an arm control device that controls the support arm,
wherein the support arm includes an actuator that drives the support arm, and a sensor that measures a load applied to the actuator, and
the arm control device includes a generation unit that generates three-dimensional information in a body into which the endoscope is inserted, and a correction unit that corrects the three-dimensional information on a basis of the measured load.
